(19) United States
(12) Patent Application Publication
(10) Pub. No.: US 2013/0178441 A1
(43) Pub. Date: Jul. 11, 2013

(54) Methods for Extending Progression-Free Survival using 10-Propargyl-10-Deazaaminopterin

(71) Applicant: Steven M. Fruchtman, New York, NY (US)

(72) Inventor: Steven M. Fruchtman, New York, NY (US)

(73) Assignee: ALLOS THERAPEUTICS, INC., Westminster, CO (US)

(21) Appl. No.: 13/816,157

(22) PCT Filed: Aug. 5, 2011
(86) PCT No.: PCT/US11/46711

§ 371 Date: Mar. 20, 2013

Related U.S. Application Data
(60) Provisional application No. 61/372,377, filed on Aug. 10, 2010.
(60) Provisional application No. 61/419,713, filed on Dec. 3, 2010.

(51) Int. Cl. A61K 31/519; A61K 31/7052

(52) U.S. Cl. CPC A61K 31/519; A61K 31/7052
USPC 514/52; 514/249

(57) ABSTRACT

The present invention includes a method to reverse the trend towards progressive resistance to consecutive treatments for T-cell lymphoma in a patient. This method includes selecting a patient having T-cell lymphoma, wherein the patient has had at least one prior treatment for T-cell lymphoma, wherein the prior treatment comprises non-10-propargyl-10-deazaaminopterin; and administering to the patient a composition comprising a therapeutically effective amount of 10-propargyl-10-deazaminopterin, whereby the trend towards progressive resistance is reversed.

### TECHNICAL FIELD

The present invention relates to methods to treat cancer with 10-propargyl-10-deazaminopterin.

### BACKGROUND OF THE INVENTION

10-propargyl-10-deazaminopterin (encompassing “10-propargyl-10-dAM,” “pralatrexate,” “racemic PDX,” “(2S)-2-[[4-[(1RS)-1-[(2,4-diaminopteridin-6-yl)methyl]but-3-ynyl]benzoyl]amino]pentanedioic acid,” “(2RS)-2-[[4-[(1RS)-1-[(2,4-diaminopteridin-6-yl)methyl]but-3-ynyl]benzoyl]amino]pentanedioic acid,” and “PDX”), is a compound which has been tested and found useful in the treatment of cancer. 10-propargyl-10-deazaminopterin has been approved by the U.S. Food and Drug Administration (FDA) as a treatment for relapsed and refractory peripheral T-cell lymphoma. 10-propargyl-10-deazaminopterin is also being investigated for use in lymphoma, lung cancer, bladder cancer, and breast cancer.

10-propargyl-10-deazaminopterin was originally disclosed by DeGraw et al., “Synthesis and Antitumor Activity of 10-Propargyl-10-deazaminopterin,” J. Med. Chem. 36: 2228-2231 (1993).

U.S. Pat. No. 6,028,071 and PCT Publication No. WO 1998/02163, disclose that highly purified 10-propargyl-10-deazaminopterin compositions when tested in a xenograft model have efficacy against human tumors. Subsequent studies with 10-propargyl-10-deazaminopterin have shown that it is useful on its own and in combinations with other therapeutic agents. For example, Sirotnak et al., Clinical Cancer Research Vol. 6, 3705-3712 (2000) reports that co-administration of 10-propargyl-10-deazaminopterin and probenecid, an inhibitor of a cMOAT/MRP-like plasma membrane ATPase, greatly enhances the efficacy of 10-propargyl-10-deazaminopterin against human solid tumors. 10-propargyl-10-deazaminopterin and combinations of 10-propargyl-10-deazaminopterin with platinum based chemotherapeutic agents have been shown to be effective against mesothelioma. (Khokar, et al., Clin. Cancer Res. 7: 3199-3205 (2001). Co-administration with gemcitabine (Gem), for treatment of lymphoma, has been disclosed in WO/2005/117892. Combinations of 10-propargyl-10-deazaminopterin with taxols are disclosed to be efficacious in U.S. Pat. No. 6,323,205. 10-propargyl-10-deazaminopterin has also shown to be effective for treatment of T-cell lymphoma, see U.S. Pat. No. 7,622,470. Other studies have shown a method for assessing sensitivity of a lymphoma to treatment with 10-propargyl-10-deazaminopterin by determining the amount of reduced folate carrier-1 protein (RFC-1) expressed by the sample, wherein a higher level of expressed RFC-1 is indicative of greater sensitivity to 10-propargyl-10-deazaminopterin, disclosed in PCT Publication No. WO 2005/117892.

10-propargyl-10-deazaminopterin is known as an antifolate/antimetabolite. Several proteins are implicated in the metabolism of folic acid and as targets of anti-folates such as 10-propargyl-10-deazaminopterin and methotrexate (MTX) in tumor cells.

A number of publications have reported on a decrease in efficacy with consecutive different treatments for malignant disease. Treatment-naive patients show a greater response to chemotherapy than patients receiving second line therapy in a wide range of malignancies, including non-Hodgkin's lymphoma (NHL) (Alici et al., 2006; Marcus, 2007; Sammler et al., 2008; Schein et al., 1975; Sehouli et al., 2002). Furthermore, in conditions where survival is long enough to undergo more than two different treatments, remission has been shown to shorten with each treatment regimen until resistance occurs. This has been seen in conditions as diverse as indolent lymphoma and breast cancer (Berruti et al., 2000; Johnson et al., 1995; Vignot et al., 2007).

Pralatrexate was approved under the provisions of accelerated approval is the United States for the treatment of patients with relapsed/refractory PTCL based on the results of the pivotal trial PROPEL (Pralatrexate in Patients with Relapsed Or Refractory Peripheral T-cell Lymphoma). The primary endpoint, objective response rate (ORR) by independent central review using International Workshop Criteria, was 29% (32/109), including 11% complete response/complete response unconfirmed (CR/CRu) with a median duration of response of 10.1 months. The ORR by investigator assessment was 39% (43/109), including 18% CR/CRu.

One of the continued problems with therapy in cancer patients, and particularly with T-cell lymphoma patients, is where survival is long enough to undergo more than two different treatments, response time has been shown to shorten with each treatment regimen until resistance occurs. In other words, where resistance is noticed to a particular therapy and a new, different therapy is started as a subsequent treatment, progression-free survival will be reduced in the subsequent treatment, and in each subsequent treatment thereafter. Therefore, a need still exists in the art for improved methods to extend progression-free survival in second-line and subsequent treatment regimens for T-cell lymphoma, among other cancers.

### BRIEF DESCRIPTION OF THE DRAWINGS

FIG. 1 shows a synthetic scheme useful in preparing 10-propargyl-10-deazaminopterin.

FIG. 2 shows a comparison of median progression free survival and objective response rate with previous lines of therapy in patients with PTCL (N=57).

FIG. 3 shows a comparison of median progression free survival and objective response rates in patients with previous lines of therapy in patients with PTCL (N=86).

### SUMMARY OF THE INVENTION

In one embodiment, the present invention includes a method to reverse the trend towards progressive resistance to consecutive treatments for T-cell lymphoma in a patient, comprising: selecting a patient having T-cell lymphoma, wherein the patient has had at least one prior treatment for T-cell lymphoma, wherein the at least one prior treatment comprises a non-10-propargyl-10-deazaminopterin treatment; and administering to the patient a composition comprising a therapeutically effective amount of 10-propargyl-10-deazaminopterin, whereby the trend towards progressive resistance is reversed.

In one embodiment, the trend towards reversal of progressive resistance to consecutive treatments for T-cell lymphoma in a patient is measured by extension of the number of days of the patient's progression-free survival upon treatment with a composition comprising 10-propargyl-10-deazaminopterin, relative to the patient's number of days of progression-free survival for the immediately-prior non-10-propargyl-10-deazaminopterin treatment.

In one embodiment, the 10-propargyl-10-deazaminopterin is substantially free of 10-deazaminopterin. The present invention also relates to methods where the T-cell lymphoma is peripheral T-cell lymphoma, or cutaneous T-cell lymphoma, or a subcutaneous panniculitic T-cell lymphoma, or a human T-lymphotropic virus (HTLV)-associated T-cell lymphoma/leukemia. In other embodiments, the T-cell lymphoma may include (a) lymphoblastic lymphomas in which the malignancy occurs in primitive lymphoid progenitors from the thymus; (b) mature or peripheral T cell neoplasms, including T cell prolymphocytic leukemia, T-cell granular lymphocytic leukemia, aggressive NK-cell leukemia, cutaneous T cell lymphoma (Mycosis fungoides/Sezary syndrome), anaplastic large cell lymphoma, T cell type, enteropathy-type T cell lymphoma, Adult T-cell leukemia/lymphoma including those associated with HTLV-1, and angioimmunoblastic T cell lymphoma, and subcutaneous panniculitic T cell lymphoma; and (c) peripheral T cell lymphomas that initially involve a lymph node paracortex and never grow into a true follicular pattern. In some embodiments, the T-cell lymphoma is relapsed/refractory T-cell lymphoma.

In some embodiments, the 10-propargyl-10-deazaminopterin is in a salt form. The 10-propargyl-10-deazaminopterin may be administered in an amount of from 30 to 275 mg/m² per dose; may be administered weekly. In some embodiments, the composition comprising 10-propargyl-10-deazaminopterin is administered in an amount of 30 mg/m² per dose. In some embodiments, the composition comprising 10-propargyl-10-deazaminopterin is administered biweekly. The composition comprising 10-propargyl-10-deazaminopterin may also badministered in one or more cycles, each cycle comprising administration once weekly for six weeks in an amount of from 30 to 150 mg/m2 per dose followed by a one week rest. The composition may be administered as an oral liquid or as an injectable solution.

In some embodiments, administration of the composition comprising 10-propargyl-10-deazaminopterin further comprises supplementation with folic acid and vitamin B12.

In some embodiments, the patient has had one prior non-10-propargyl-10-deazaminopterin treatment. In other embodiments, the patient has had two prior non-10-propargyl-10-deazaminopterin treatments. In other embodiments, the patient has had three prior non-10-propargyl-10-deazaminopterin treatments. Other embodiments include where the patient has had greater than three prior non-10-propargyl-10-deazaminopterin treatments. In some embodiments, the at least one prior non-10-propargyl-10-deazaminopterin treatment comprises cyclophosphamide, doxorubicin, vincristine, and prednisone (CHOP). In other embodiments, the at least one prior non-10-propargyl-10-deazaminopterin treatment comprises ifosfamide, carboplatin, and etoposide (ICE).

### DETAILED DESCRIPTION OF THE INVENTION

One of the continued problems with therapy in cancer patients is acquired resistance to a particular type of therapy. Cancer cells can acquire resistance to chemotherapy by a range of mechanisms, including the mutation or overexpression of the drug target, inactivation of the drug, or elimination of the drug from the cell. Typically, tumors will then recur. In the conventional view of drug resistance, one or several cells in the tumor population may acquire genetic changes that confer drug resistance. These cells have a selective advantage that allows them to overtake the population of tumor cells following cancer chemotherapy. Based on the tumor-stem-cell concept, an alternative model posits that the cancer stem cells are naturally resistant to chemotherapy through their quiescence, their capacity for DNA repair, and ABC-transporter expression. As a result, at least some of the tumor stem cells can survive chemotherapy and support regrowth of the tumor. A new treatment can then be administered, but the tumor will often acquire resistance to the new or “second-line” treatment. Depending on the health of the patient, third-line, fourth-line and so on types of treatments can then be administered upon acquired resistance to each immediately-prior treatment.

A cancer is “responsive” to a therapeutic agent or there is a “good response” to a treatment if its rate of growth is inhibited as a result of contact with the therapeutic agent, compared to its growth in the absence of contact with the therapeutic agent. Growth of a cancer can be measured in a variety of ways, for instance, the size of a tumor or the expression of tumor markers appropriate for that tumor type may be measured. These criteria define the type of response measured and also the characterization of time to disease progression which is another important measure of a tumor's sensitivity to a therapeutic agent. Still further, measures of responsiveness can be assessed using additional criteria beyond growth size of a tumor, including patient quality of life, degree of metastases, etc. In addition, clinical prognostic markers and variables can be assessed in applicable situations.

A cancer is “non-responsive” or has a “poor response” to a therapeutic agent such as 10-propargyl-10-deazaminopterin or there is a poor response to a treatment if its rate of growth is not inhibited, or inhibited to a very low degree, as a result of contact with the therapeutic agent when compared to its growth in the absence of contact with the therapeutic agent. As stated above, growth of a cancer can be measured in a variety of ways, for instance, the size of a tumor or the expression of tumor markers appropriate for that tumor type may be measured. The quality of being non-responsive to a therapeutic agent is a highly variable one, with different cancers exhibiting different levels of “non-responsiveness” to a given therapeutic agent, under different conditions. Still further, measures of non-responsiveness can be assessed using additional criteria beyond growth size of a tumor, including patient quality of life, degree of metastases, etc. In addition, clinical prognostic markers and variables can be assessed in applicable situations.

Progression-free survival or PFS is a term used to describe the length of time during and after medication or treatment during which the disease being treated (usually cancer) does not get worse. It is sometimes used as a metric to study health of a person with a disease to try to determine how well a new treatment is working. The time interval from the start of treatment to disease progression is the classic definition of progression-free survival. It is a measure of the clinical benefit from therapy. PFS is a metric frequently used to evaluate the cost effectiveness of a cancer treatment. PFS has been postulated by several key opinion leaders in oncology to be a better (“more pure”) measure of efficacy in second-line clinical trials as it eliminates potential differential bias from prior or subsequent treatments.

One of the continued problems with therapy in cancer patients, and particularly with T-cell lymphoma patients, is where survival is long enough to undergo more than 2 different treatments, response time has been shown to shorten with each treatment regimen until resistance occurs. In other words, where resistance is noticed to a particular treatment and a new, different treatment is started as a subsequent treatment, progression-free survival will be reduced in the subsequent treatment, and in each subsequent treatment thereafter. This is a problem well known in the art. Progressive resistance can be seen in analyses in patients' response and PFS to prior treatments using patients as their own control. The expected and normally-observed result is that response rates and PFS will be lower with each subsequent line of treatment.

According to the instant invention, when analyses were conducted on those patients who received at least 1 prior treatment(s) prior to 10-propargyl-10-deazaminopterin treatment, this trend towards reversal of the trend towards reduction in number of days of progression-free survival was observed. PFS and response rate of third prior treatment (−3) was compared with those of the second prior treatment (−2), PFS and response rate of second prior treatment (−2) was compared with those last line of treatment (−1) prior to 10-propargyl-10-deazaminopterin, and PFS and response rate of last line of treatment (−1) was compared with 10-propargyl-10-deazaminopterin treatment for these patients. Surprisingly, analysis according to the instant invention demonstrated that rather than progressive resistance, 10-propargyl-10-deazaminopterin given as second-line, third-line or later treatment reversed this trend. The analysis was conducted on all patients comparing PFS and response rate of last line of treatment with those of 10-propargyl-10-deazaminopterin treatment.

Progressive resistance to consecutive treatments can refer to reduction in the number of days of progression-free survival obtained upon each consecutive treatment for a disease, relative to the number of days of progression-free survival for the previous treatment. In other words, the longest period of progression-free survival will be obtained for the first treatment of a disease, the second longest period of progression-free survival will be obtained for the second treatment of a disease, the third longest period of progression-free survival will be obtained for the third treatment of a disease, and so forth. As discussed herein, it is known in the art that the number of days of progression-free survival is reduced for each new treatment relative to the number of days of progression-free survival for the previous treatment. As discussed, this art-known tendency for reduction in the number of days of progression-free survival per each consecutive treatment may be referred to herein as “progressive resistance to consecutive treatments.” Such progressive resistance may be observed after two consecutive treatments, three consecutive treatments, four consecutive treatments, five consecutive treatments, six consecutive treatments, or more.

Reversal of a trend towards progressive resistance or reversal of progressive resistance may include an observation that the number of days of progression-free survival upon treatment with 10-propargyl-10-deazaminopterin are the same as or greater than the number of days of progression-free survival of a previous, non-10-propargyl-10-deazaminopterin treatment.

Generally, the consecutive treatments will each be different in some aspect from the previous or following treatments, but in some embodiments the consecutive treatments can be the same or consecutive treatments may include the same treatment more than once. For example, a first treatment will commonly comprise a chemotherapy treatment with a given chemotherapy agent or combination of chemotherapy agents. A second treatment may include a chemotherapy with a given chemotherapy agent different from the chemotherapy agent given in the first treatment, or a different combination of chemotherapy agents than given in the first treatment. The combination given in the second treatment may or may not include an agent that was given in the first treatment, either alone or as part of a combination. The combination given in the third treatment may or may not include an agent that was given in the first or second treatments, either alone or as part of a combination. The combination given in the fourth treatment may or may not include an agent that was given in the first, second, or third treatments, either alone or as part of a combination. The combination given in the fifth treatment may or may not include an agent that was given in the first, second, third or fourth treatments, either alone or as part of a combination. If any consecutive treatments are given past the fifth treatment, the same principles apply to those treatments as are enumerated above.

As used herein, “treatment” can mean the use of a therapy to prevent or inhibit further tumor growth, as well as to cause shrinkage of a tumor, and to provide longer survival times. Treatment is also intended to include prevention of metastasis of tumor. A tumor is “inhibited” or “treated” if at least one symptom (as determined by responsiveness/non-responsiveness, time to progression, or indicators known in the art and described herein) of the cancer or tumor is alleviated, terminated, slowed, minimized, or prevented. Any amelioration of any symptom, physical or otherwise, of a tumor pursuant to treatment using a therapeutic regimen (e.g., 10-propargyl-10-deazaminopterin) as further described herein, is within the scope of the invention.

A treatment according to the instant invention may consist of any therapy known in the art for treating cancer, and includes, without limitation, such treatments or therapies such as chemotherapy, radiotherapy, and/or combinations of the above. Chemotherapy can refer to chemical treatment to kill or halt the replication and/or spread of cancerous cells in a patient, and may involve one agent, or a combination of more than one agent. Any treatment known in the art appropriate for cancer may be used in the present invention and is appropriate as a prior treatment (prior to pralatrexate). In one embodiment, a treatment or prior treatment is a treatment for T-cell lymphoma. Appropriate prior treatments or treatments for T-cell lymphoma include, without limitation, a treatment comprising the multi-agent combination cyclophosphamide, doxorubicin, vincristine, and prednisone (CHOP); a treatment comprising the multi-agent combination rituximab, cyclophosphamide, doxorubicin, vincristine, prednisolone (R-CHOP); a treatment comprising the multi-agent combination ifosfamide, carboplatin, and etoposide (ICE); a treatment comprising the multi-agent combination derived from ICE-based regimens (rituximab-ICE [RICE]); a treatment comprising the multi-agent combination dexamethasome-ICE [DICE]; a treatment comprising the multiagent combination etoposide, ifosfamide, cisplatin, and dexamethasone (VIPD); a treatment comprising the multi-agent combination etoposide, ifosfamide, methotrexate, dexamethasone, and asparaginase (SMILE).

In some embodiments, the patient has had one prior non-10-propargyl-10-deazaminopterin treatment. In other embodiments, the patient has had two prior non-10-propargyl-10-deazaminopterin treatments. In other embodiments, the patient has had three prior non-10-propargyl-10-deazaminopterin treatments. Other embodiments include where the patient has had greater than three prior non-10-propargyl-10-deazaminopterin treatments. In some embodiments, the at least one prior non-10-propargyl-10-deazaminopterin treatment comprises CHOP. In other embodiments, the at least one prior non-10-propargyl-10-deazaminopterin treatment comprises ICE.

In one aspect, the present invention includes a method to reverse the trend towards progressive resistance to consecutive treatments for T-cell lymphoma in a patient, comprising: selecting a patient having T-cell lymphoma, wherein the patient has had at least one prior treatment for T-cell lymphoma, wherein the prior treatment comprises non-10-propargyl-10-deazaminopterin treatment; and administering to the patient a composition comprising a therapeutically effective amount of 10-propargyl-10-deazaminopterin, whereby the trend towards progressive resistance is reversed.

In one embodiment, the trend towards reversal of progressive resistance to consecutive treatments for T-cell lymphoma in a patient is measured by extension of the number of days of the patient's progression-free survival upon treatment with a composition comprising 10-propargyl-10-deazaminopterin, relative to the patient's number of days of progression-free survival for a prior non-10-propargyl-10-deazaminopterin treatment. In one embodiment, the prior non-10-propargyl-10-deazaminopterin is the immediately-prior prior non-10-propargyl-10-deazaminopterin treatment.

10-propargyl-10-deazaminopterin contains asymmetric centers at carbon 10 (C10) and carbon 19 (C19) positions. In one embodiment, 10-propargyl-10-deazaminopterin includes an approximately 1:1 racemic mixture of the R- and S-configurations at the C10 chiral center, and ≧98.0% of the S-diastereomer at the C19 chiral center. 10-propargyl-10-deazaminopterin includes the C10 diastereomers PDX-10a [S-configuration] Chemical name: (2S)-2-[[4-[(1S)-1-[(2,4-diaminopteridin-6-yl)methyl]but-3-ynyl]benzoyl]amino]pentanedioic acid, and PDX-10b [R-configuration] Chemical name: (2S)-2-[[4-[(1R)-1-[(2,4-diaminopteridin-6-yl)methyl]but-3-ynyl]benzoyl]amino]pentanedioic acid.

10-propargyl-10-deazaminopterin can be synthesized using the method disclosed in Example 7 of DeGraw et al., U.S. Pat. No. 5,354,751, which is directed to manufacturing 10-propargyl-10-deazaminopterin, is incorporated by reference herein in its entirety. 10-propargyl-10-deazaminopterin may also be synthesized by methods presented in U.S. Pat. No. 6,028,071, especially in Example 1, which example is incorporated by reference herein. In some embodiments, 10-propargyl-10-deazaminopterin may be “substantially pure” 10-propargyl-10-deazaminopterin according to U.S. Pat. No. 6,028,071.

In order to generate diastereomers of 10-propargyl-10-deazaminopterin, 10-propargyl-10-deazaminopterin may be synthesized as taught herein and elsewhere, and either the final product or an earlier intermediate product may be subsequently used as a starting material to separate the C10 diastereomers. Alternately, a chiral synthesis may be employed where PDX-10a and/or PDX-10b is produced directly from any of a number of starting materials. Chiral columns to separate enantiomers or diastereomers, known in the art, may be employed to separate the diastereomers of the final 10-propargyl-10-deazaminopterin or an earlier intermediate. Suitable chiral columns for separating the diastereomers include the chiral column CHIRALPAK AD, available from Daicel Chemical Industries Ltd., Japan, using ethanol as the mobile phase.

10-propargyl-10-deazaminopterin can be administered in a wide variety of different dosage forms. For example, the 10-propargyl-10-deazaminopterin can preferably be administered orally or parenterally. In one embodiment, the 10-propargyl-10-deazaminopterin can be administered orally. In one embodiment, 10-propargyl-10-deazaminopterin is administered parenterally, and may be administered via the intravenous route.

The 10-propargyl-10-deazaminopterin can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, and others. Oral pharmaceutical compositions can be suitably sweetened and/or flavored. For oral administration of 10-propargyl-10-deazaminopterin, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the 10-propargyl-10-deazaminopterin may be combined with various sweetening or flavoring agents, coloring matter or dyes, and, if so desired, emulsifying and/or suspending agents as well, together with such diluents as water, ethanol, propylene glycol, glycerin and various like combinations thereof. A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 10 g of the active ingredient and each cachet or capsule preferably containing from about 0.05 mg to about 10 g of the active ingredient; tablets may also suitably contain about 2.5 mg active ingredient per tablet or about 7.5 mg per tablet.

For parenteral administration of 10-propargyl-10-deazaminopterin, solutions may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art.

For veterinary purposes, the active agents can be administered separately or together to animals using any of the forms and by any of the routes described above. In a preferred embodiment, 10-propargyl-10-deazaminopterin is administered in the form of a capsule, bolus, tablet, liquid drench, by injection or as an implant. As an alternative, the 10-propargyl-10-deazaminopterin can be administered with the animal feedstuff, and for this purpose a concentrated feed additive or premix may be prepared for a normal animal feed. Such formulations are prepared in a conventional manner in accordance with standard veterinary practice.

In one embodiment the composition is comprised of a pharmaceutically acceptable carrier and a therapeutically effective amount of 10-propargyl-10-deazaminopterin (including pharmaceutically acceptable salts esters, solvates, and polymorphs of each component thereof). Moreover, within this embodiment, the invention encompasses a pharmaceutical composition for the treatment of disease, the use of which results in the inhibition of growth of neoplastic cells, benign or malignant tumors, or metastases, or treatment of inflammation, comprising a pharmaceutically acceptable carrier and a non-toxic therapeutically effective amount of 10-propargyl-10-deazaminopterin (including pharmaceutically acceptable salts thereof).

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. In one embodiment, the salt is the hydrochloride salt. Salts derived from pharmaceutically acceptable organic non-toxic bases also include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

10-propargyl-10-deazaminopterin according to the present invention will typically be administered to the patient in a dose regimen that provides for the most effective treatment (from both efficacy and safety perspectives) for which the patient is being treated, as known in the art. In conducting the treatment method of the present invention, the 10-propargyl-10-deazaminopterin for use in a treatment according to the present invention can be administered in any effective manner known in the art, such as by oral, topical, intravenous, intra-peritoneal, intramuscular, intra-articular, subcutaneous, intranasal, intra-ocular, vaginal, rectal, intracranial, or intradermal routes, depending upon the type of cancer being treated, and the medical judgment of the prescribing physician as based, e.g., on the results of published clinical studies.

10-propargyl-10-deazaminopterin for use in a treatment according to the present invention can be formulated as part of a pharmaceutical preparation. The specific dosage form will depend on the method of administration, but may include tablets, capsules, oral liquids, and injectable solutions for oral, intravenous, intramuscular, intracranial, or intraperitoneal administration, and the like. Dosing may be expressed as mg/m². Alternatively, dosing may be expressed as mg/kg body weight by any manner acceptable to one skilled in the art. One method for obtaining an equivalent dosing in mg/kg body weight involves applying the conversion factor 0.025 mg/kg, for an average human, as approximately equivalent to 1 mg/m². According to this calculation, dosing of 150 mg/m² is approximately equivalent to about 3.75 mg/kg.

Appropriate dosing for oncology includes the following dosage regimes. For example, doses on the order of 10 to 120 mg/m² of body surface area/day (about 0.25 to 3 mg/kg body weight per day) are appropriate. Dosages of 30 mg/m² (about 0.75 mg/kg) once weekly for 3 weeks followed by a one week rest, 30 mg/m² (about 0.75 mg/kg) once weekly×6 weeks followed by a one week rest, or gradually increasing doses of 10-propargyl-10-deazaminopterin on the once weekly×6 week schedule are also suitable. Lower doses may be used as appropriate based on patient tolerance and type of malignancy. Higher doses can be utilized where less frequent administration is used. Thus, in a general sense, dosages of 10 to 275 mg/m² (about 0.25 to about 6.9 mg/kg) are suitably used with various dosing schedules, for example between about 100 to 275 mg/m² (about 2.5 to about 6.87 mg/kg) for biweekly dosages, and between about 10 to 150 mg/m² (about 0.25 to about 3.75 mg/kg), or, more specifically, between about 10 and 60 mg/m² for once weekly dosages.

The determination of suitable dosages using protocols similar to those described in U.S. Pat. No. 6,323,205 is within the skill in the art. In one embodiment, 10-propargyl-10-deazaminopterin for use in a treatment according to the present invention can be administered in an amount of from about 10 to about 275 mg/m² (about 0.25 to about 6.87 mg/kg) per dose. Methods of the present invention also include administration of 10-propargyl-10-deazaminopterin for use in a treatment according to the present invention weekly; in a dose of about 10 mg/m² (0.25 mg/kg) or about 30 mg/m² (0.75 mg/kg); in an amount of from about 10 to about 150 mg/m² (about 0.25 to about 3.75 mg/kg) per dose; biweekly; and in a dosage amount of about 100 to about 275 mg/m²(about 2.5 to about 6.9 mg/kg). In one embodiment, 10-propargyl-10-deazaminopterin for use in a treatment according to the present invention can be administered in an amount of between about 0.25 mg/kg and about 4 mg/kg; between about 0.75 mg/kg and about 3 mg/kg; in an amount between about 1.0 mg/kg and about 2.5 mg/kg; in an amount of about 0.25 mg/kg or about 0.75 mg/kg (or an equivalent amount in body surface area (BSA)).

10-propargyl-10-deazaminopterin may be used in combinations with other cytotoxic and antitumor compounds, including vinca alkaloids such as vinblastine, navelbine, and vindesine; probenicid, nucleotide analogs such as gemcitabine, 5-fluorouracil, and cytarabine; alkylating agents such as cyclophosphamide or ifosfamide; cisplatin or carboplatin; leucovorin; taxanes such a paclitaxel or docetaxel; anti-CD20 monoclonal antibodies, with or without radioisotopes, and antibiotics such as doxorubicin and mitomycin. Combinations of 10-propargyl-10-deazaminopterin with several of these other antitumor agents or with growth factor inhibitors and anti-angiogenic agents may also be used.

10-propargyl-10-deazaminopterin and other agents may be concurrently administered or utilized in combination as part of a common treatment regimen, in which the 10-propargyl-10-deazaminopterin and the other agent(s) are administered at different times. For example, the other agent may be administered before, immediately afterward or after a period of time (for example 24 hours) relative to the 10-propargyl-10-deazaminopterin administration. Thus, for purposes of this application, the term administering refers generally to concurrent administration or to sequential administration of the drugs and in either order in a parallel treatment regimen with or without a separation in time between the drugs unless otherwise specified.

10-propargyl-10-deazaminopterin is suitably used in combination with folic acid and vitamin B12 supplementation to reduce the side effects of the treatment. For example, patients may be treated with folic acid (1 mg/m² daily starting 1 week prior to treatment with 10-propargyl-10-deazaminopterin, or alternatively 1 mg perioral (p.o.) daily not based on body surface area (BSA)); and B 12 (1 mg/m² monthly, or alternatively given intramuscularly (I.M.) every 8-10 weeks as 1 mg (not based on BSA), or alternatively p.o. daily 1 mg (not based on BSA)).

As utilized herein, the “subject” or “patient” of the methods described herein can be any animal. In a preferred embodiment, the animal of the present invention is a human. In addition, determination of expression patterns is also contemplated for non-human animals which can include, but are not limited to, cats, dogs, birds, horses, cows, goats, sheep, guinea pigs, hamsters, gerbils, mice and rabbits.

The term “cancer,” when used herein refers to or describes the pathological condition, preferably in a mammalian subject, that is typically characterized by unregulated cell growth. Non-limiting cancer types include carcinoma (e.g., adenocarcinoma), sarcoma, myeloma, leukemia, and lymphoma, and mixed types of cancers, such as adenosquamous carcinoma, mixed mesodermal tumor, carcinosarcoma, and teratocarcinoma.

In one embodiment, cancers include solid tumors, in particular, non-small cell lung cancer, head and neck cancer, prostate cancer, and breast cancer. Other cancers include but are not limited to, bladder cancer, lung cancer, colon cancer, rectal cancer, endometrial cancer, ovarian cancer; and melanoma. Specifically included are AIDS-related cancers (e.g., Kaposi's Sarcoma, AIDS-related lymphoma), bone cancers (e.g., osteosarcoma, malignant fibrous histiocytoma of bone, Ewing's Sarcoma, and related cancers), and hematologic/blood cancers (e.g., adult acute lymphoblastic leukemia, childhood acute lymphoblastic leukemia, adult acute myeloid leukemia, childhood acute myeloid leukemia, chronic lymphocytic leukemia, chronic myelogenous leukemia, hairy cell leukemia, adult Hodgkin's disease, childhood Hodgkin's disease, Hodgkin's disease during pregnancy, adult non-Hodgkin's lymphoma, childhood non-Hodgkin's lymphoma, non-Hodgkin's lymphoma during pregnancy, primary central nervous system lymphoma, Waldenstrom's macroglobulinemia, multiple myeloma/plasma cell neoplasm, myelodysplastic syndrome, and myeloproliferative disorders).

In one embodiment, the cancer includes T-cell lymphoma. The T-cell lymphoma can include peripheral T-cell lymphoma (PTCL), or cutaneous T-cell lymphoma (CTCL). In one embodiment the T-cell lymphoma is relapsed and/or refractory T-cell lymphoma. Other T-cell lymphomas to treat include lymphoblastic lymphomas in which the malignancy occurs in primitive lymphoid progenitors from the thymus; mature or peripheral T-cell neoplasms, including T-cell prolymphocytic leukemia, T-cell granular lymphocytic leukemia, aggressive NK-cell leukemia, cutaneous T-cell lymphoma (Mycosis fungoides/Sezary syndrome), anaplastic large cell lymphoma, T-cell type, enteropathy-type T-cell lymphoma, Adult T-cell leukemia/lymphoma including those associated with HTLV-1, and angioimmunoblastic T-cell lymphoma, and subcutaneous panniculitic T-cell lymphoma; and peripheral T-cell lymphomas that initially involve a lymph node paracortex and never grow into a true follicular pattern. In some embodiments, the T-cell lymphoma is relapsed/refractory T-cell lymphoma.

Also included are brain cancers (e.g., adult brain tumor, childhood brain stem glioma, childhood cerebellar astrocytoma, childhood cerebral astrocytoma, childhood ependymoma, childhood medulloblastoma, supratentorial primitive neuroectodermal and pineal, and childhood visual pathway and hypothalamic glioma), digestive/gastrointestinal cancers (e.g., anal cancer, extrahepatic bile duct cancer, gastrointestinal carcinoid tumor, colon cancer, esophageal cancer, gallbladder cancer, adult primary liver cancer, childhood liver cancer, pancreatic cancer, rectal cancer, small intestine cancer, and gastric cancer), musculoskeletal cancers (e.g., childhood rhabdomyosarcoma, adult soft tissue sarcoma, childhood soft tissue sarcoma, and uterine sarcoma), and endocrine cancers (e.g., adrenocortical carcinoma, gastrointestinal carcinoid tumor, islet cell carcinoma (endocrine pancreas), parathyroid cancer, pheochromocytoma, pituitary tumor, and thyroid cancer).

Also included are neurologic cancers (e.g., neuroblastoma, pituitary tumor, and primary central nervous system lymphoma), eye cancers (e.g., intraocular melanoma and retinoblastoma), genitourinary cancers (e.g., bladder cancer, kidney (renal cell) cancer, penile cancer, transitional cell renal pelvis and ureter cancer, testicular cancer, urethral cancer, Wilms' tumor and other childhood kidney tumors), respiratory/thoracic cancers (e.g., non-small cell lung cancer, small cell lung cancer, malignant mesothelioma, and malignant thymoma), germ cell cancers (e.g., childhood extracranial germ cell tumor and extragonadal germ cell tumor), skin cancers (e.g., melanoma, and merkel cell carcinoma), gynecologic cancers (e.g., cervical cancer, endometrial cancer, gestational trophoblastic tumor, ovarian epithelial cancer, ovarian germ cell tumor, ovarian low malignant potential tumor, uterine sarcoma, vaginal cancer, and vulvar cancer), and unknown primary cancers.

Unless otherwise indicated, all numbers expressing quantities of ingredients, dimensions reaction conditions and so forth used in the specification and claims are to be understood as being modified in all instances by the term “about”.

In this application and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

The following examples are provided for illustrative purposes only and are not intended to limit the scope of the invention.

### Example 1

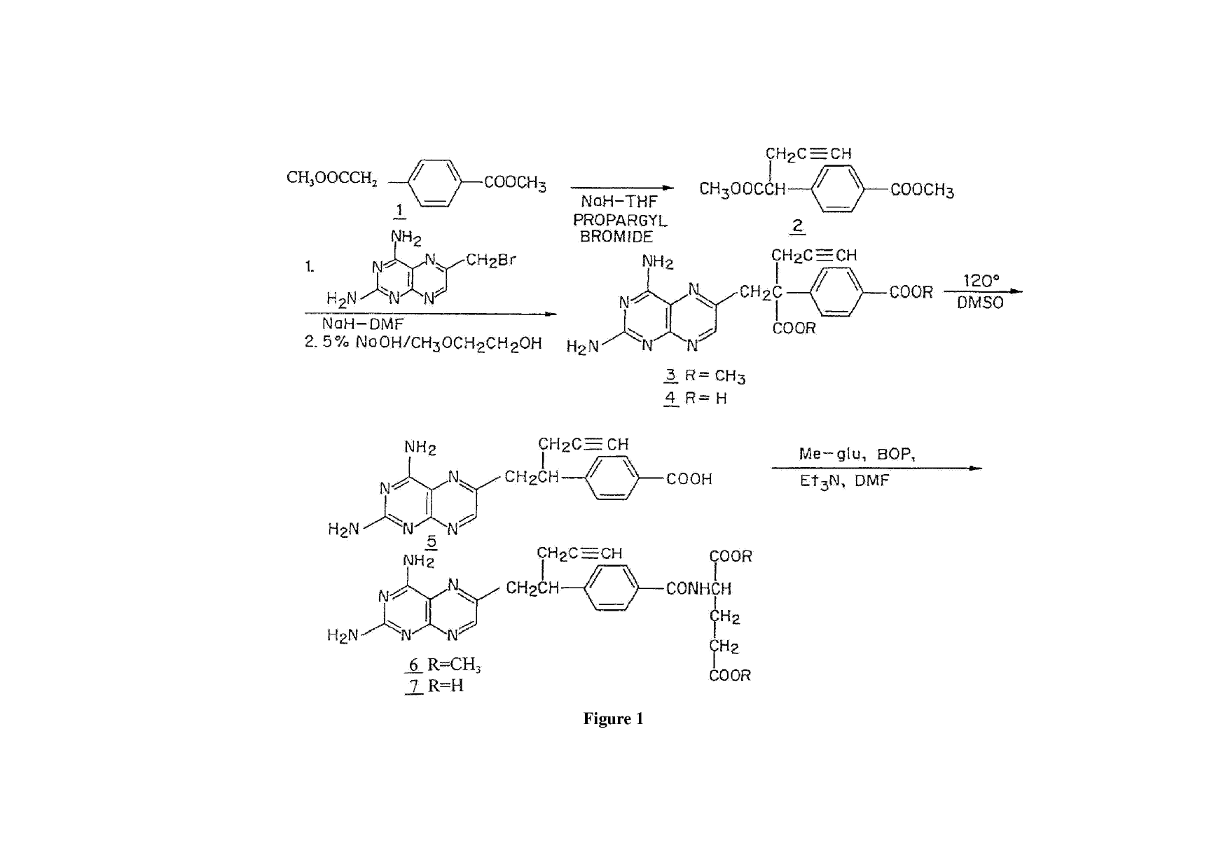

FIG. 1 shows a synthetic scheme useful in preparing 10-propargyl-10-deazaminopterin. A mixture of 60% NaH in oil dispersion (1.06 g, 26.5 mmol) in 18 mL of sieve-dried THF was cooled to 0° C. The cold mixture was treated with a solution of homoterephthalic acid dimethyl ester (5.0 g, 24 mmol compound 1 in FIG. 1) in dry THF (7 mL), and the mixture was stirred for 1 hour at 0° C. Propargyl bromide (26.4 mmol) was added, and the mixture was stirred at 0° C. for an additional 1 hour, and then at room temperature for 16 hours. The resulting mixture was treated with 2.4 mL of 50% acetic acid and then poured into 240 mL of water. The mixture was extracted with ether (2×150 mL). The ether extracts were combined, dried over Na₂SO₄, and concentrated to an orange-yellow oil. Chromatography on silica gel (600 mL of 230-400 mesh) with elution by cyclohexane-EtOAc (8:1) gave the product α-propargylhomoterephthalic acid dimethyl ester (compound 2) as a white solid (4.66) which appeared by TLC (cyclohexane-EtOAc, 3:1) to be homogeneous. Mass spectral data on this product, however, showed it to be a mixture of the desired product 2, and the dipropargylated compound. No starting material 1 was detected. HPLC shows the ratio of mono- to di-propargylated products to be about 3:1. Since the dipropargylated product, unlike compound 1, cannot produce an unwanted coproduct in the next step of the reaction, this material was suitable for conversion to compound 3. Absence of starting compound 1 in the product used to proceed in the synthesis is very important in order to avoid the sequential formation of 10-dAM during the transformations lading to the final product, because complete removal from 10-dAM from 10-propargyl-10-deazaminopterin is very difficult.

A mixture was formed by combining 0.36 g of a 60% NaH (9 mmol) in oil dispersion with 10 mL of dry DMF and cooled to 0-5° C. The cold mixture was treated drop-wise with a solution of the product of the first reaction (compound 2) (2.94 g, 12 mmol) in 10 mL dry DMF and then stirred at 0° C. for 30 minutes. After cooling to −25° C., a solution of 2,4,diamino-6-(bromomethyl)-pteridine hydrobromide-0.2 2-propanol (1.00 g, 2.9 mmol) in 10 mL dry DMF was added drop-wise while the temperature was maintained near −25° C. The temperature of the stirred mixture was allowed to rise to −10° C. over a period of 2 hours. After an additional 2 hours at −10° C., the temperature was allowed to rise to 20° C., stirring at room temperature was continued for 2 hours longer. The reaction was then adjusted to pH 7 by addition of solid CO₂, After concentration in vacuo to remove solvent, the residue was stirred with diethyl ether and the ether insoluble material was collected, washed with water, and dried in vacuo to give 1.49 g of a crude product. This crude product was dissolved in CHCl₃-MeOH (10:1) for application to a silica gel column Elution by the same solvent system afforded 10-propargyl-10-carbomethoxy-4-deoxy-4-amino-10-deazapteroic acid methyl ester (compound 3) which was homogenous to TLC in 40% yield (485 mg).

A stirred suspension of compound 3 (400 mg, 0.95 mmol) in 2-methoxyethanol (5 mL) was treated with water (5 mL) and then 10% sodium hydroxide solution (3.9 mL). The mixture was stirred as room temperature for 4 hours, during which time solution occurred. The solution was adjusted to pH 8 with acetic acid and concentrated under high vacuum. The resulting residue was dissolved in 15 mL of water and acidified to pH 5.5-5.8 resulting in formation of a precipitate. The precipitate was collected, washed with water and dried in vacuo to recover 340 mg of compound 4 (91% yield). HPLC analysis indicated a product purity of 90%.

Compound 4 (330 mg) was decarboxylated by heating in 15 mL DMSO at 115-120° C. for 10 minutes. A test by HPLC after 10 minutes confirmed that the conversion was essentially complete. DMSO was removed by distillation in vacuo (bath at 40° C.). The residue was stirred with 0.5 N NaOH to give a clear solution, Acidification to pH 5.0 with 1N HCl gave 10-propargyl-4-deoxy-4-amino-10-deazapteroic acid (compound 5) as a yellow solid in 70% yield. HPLC indicated product purity at this stage as 90%.

Compound 5 (225 mg, 0.65 mmol) was coupled with dimethyl L-glutamate hydrochloride (137 mg, 0.65 mmol) using BOP reagent (benzotriazole-1-yloxytris(dimethylamino) phosphonium hexafluorophosphate (287 mg, 0.65 mmol, Aldrich Chemical Co.) in DMF (10 mL) containing triethylamine (148 mg, 1.46 mmol). The mixture was stirred for 3 hours at 20-25° C. and then evaporated to dryness. The residue was stirred with water, and the water-insoluble crude product was collected and dried in vacuo. The crude product (350 mg) was purified by silica gel chromatography with elution by CHCl₃-MeOH (10:1) containing triethylamine (0.25% by volume) to recover 165 mg of 10-propargyl-10-deazaminopterin dimethyl ester (compound 6, 50% yield) which was homogeneous to TLC (CHCl₃-MeOH 5:1).

Compound 6 (165 mg, 0.326 mmol) was suspended in 10 mL stirred MeOH to which 0.72 mL (0.72 meq) 1N NaOH was added. Stirring at room temperature was continued until solution occurred after a few hours. The solution was kept at 20-25°. for 8 hours, then diluted with 10 mL water. Evaporation under reduced pressure removed the methanol, and the concentrated aqueous solution was left at 20-25° C. for another 24 hours. HPLC then showed the ester hydrolysis to be complete. The clear aqueous solution was acidified with acetic acid to pH 4.0 to precipitate 10-propargyl-10-deazaminopterin as a pale yellow solid, The collected, water washed and dried in vacuo product weighed 122 mg (79% yield). Assay by elemental analysis, proton NMR and mass spectroscopy were entirely consistent with the assigned structure. HPLC analysis indicated purity of 98% and established the product to be free of 10-deazaminopterin.

In this case, the amount of 10-propargyl-10-deazaminopterin (as determined by HPLC peak area) approaches 98%, and the peak corresponding to 10-deazaminopterin is not detected by the processing software although there is a minor baseline ripple in this area.

### Example 2

Analysis of progressive resistance. The treatment immediately prior to entry into clinical trial PDX-008 provides data from a heterogeneous control group reflecting clinical practice with which to compare the efficacy of pralatrexate. This dataset has the benefit of being from the same patients as those receiving pralatrexate, and so avoids inter-subject variation.

In a wide range of malignancies, including non-Hodgkin's lymphoma (NHL), treatment-naïve patients generally show a greater response to chemotherapy than patients receiving second-line or subsequent therapy. Furthermore, objective response rate (ORR) and progression-free survival (PFS) generally decrease with each subsequent line of therapy, the hallmark of acquired drug resistance. This trend would also be expected for PTCL, although there are no published studies or retrospective data analyses specifically in PTCL describing the pattern of response to successive treatments. The goal of the analysis presented here was to determine whether a trend of progressive resistance is observed in relapsed or refractory PTCL, and to identify the efficacy (PFS and ORR) or pralatrexate as a subsequent therapy relative to previous treatments. The PROPEL study is the largest data set published for relapsed/refractory PTCL to date. A part of the patient's medical history, data were collected on response and PFS in previous lines of therapy.

Methodology: In order to explore this progressive resistance concept, retrospective analyses were performed looking at patients' response and PFS to prior therapies using patients as their own control. It was hypothesized that the response rates and PFS would be lower with each subsequent line of therapy (hypothesis #1). Analyses were conducted on those patients who received at least 3 prior therapies. PFS and response rate of third prior therapy (−3) were compared with those of the second prior therapy (−2), PFS and response rate of second prior therapy (−2) were compared with those last line of therapy (−1) prior to pralatrexate, and PFS and response rate of last line of therapy (−1) were compared with pralatrexate therapy for these patients. These analyses utilized investigator assessment of PFS, and response since central review of tumor assessments on prior therapies was not available.

It was further hypothesized (hypothesis #2) that if progressive resistance was demonstrated, then pralatrexate may reverse or slow this trend, consistent with a clinical benefit for the drug. Therefore, an analysis was conducted on all patients comparing PFS and response rate of last line of therapy with those of pralatrexate therapy.

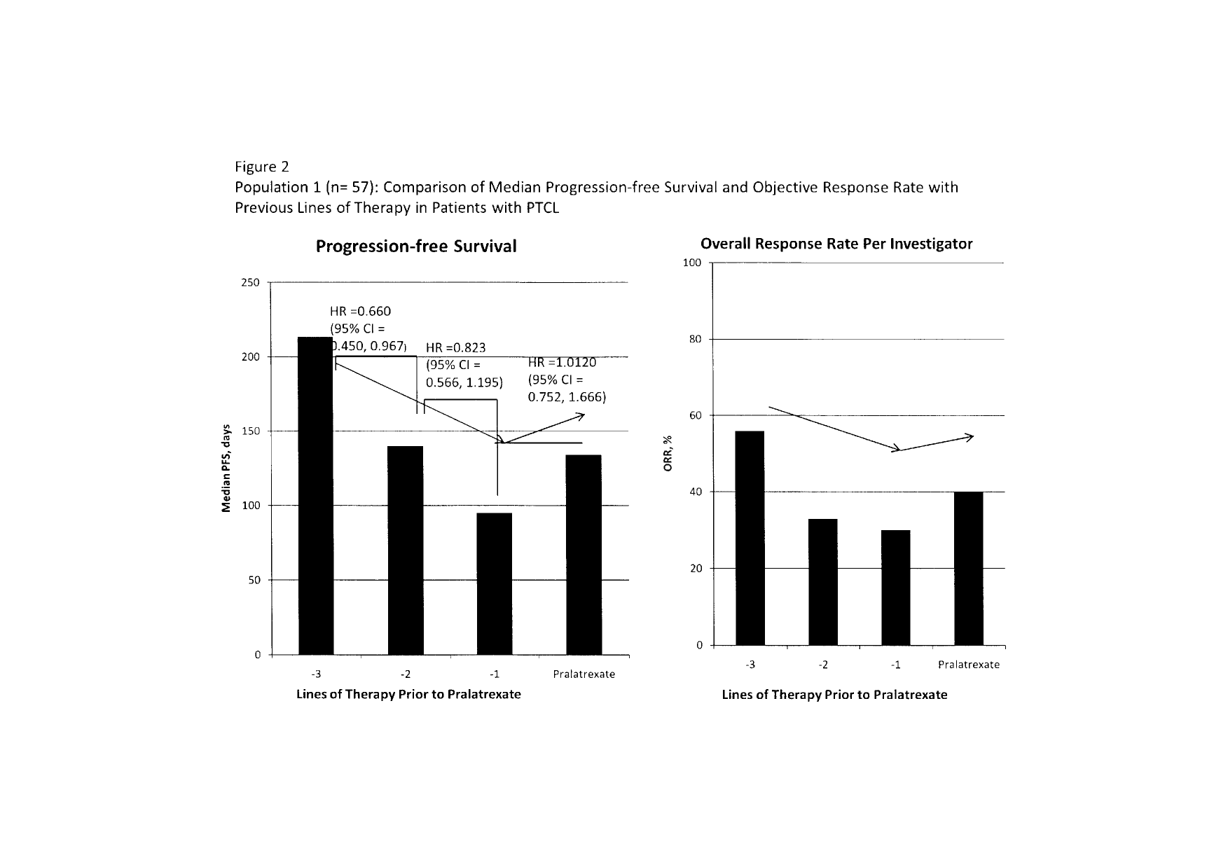

For examining hypothesis #1, there were 57 patients (−3) who had undergone at least 3 prior therapies before entry into PDX-008. Of these 57 patients, 34 had more than 3 treatments and 23 had exactly 3 previous treatments. Examination of the pattern beyond 3 previous treatments would be progressively uninformative as fewer and fewer patients would be entered into the analysis. Results are presented in Table. 1. See also FIG. 2. Analysis of the data from the 57 patients who had undergone ≧3 previous treatments demonstrated a trend for decreased PFS and ORR with each consecutive treatment (FIG. 2). This trend of decreased ORR and PFS with successive lines of therapy was reversed by pralatrexate treatment, demonstrated by a higher ORR (40%) and longer PFS (median=134 days) than the previous line of therapy [HR for −1 vs. pralatrexate=1.120 (0.752, 1.666)].

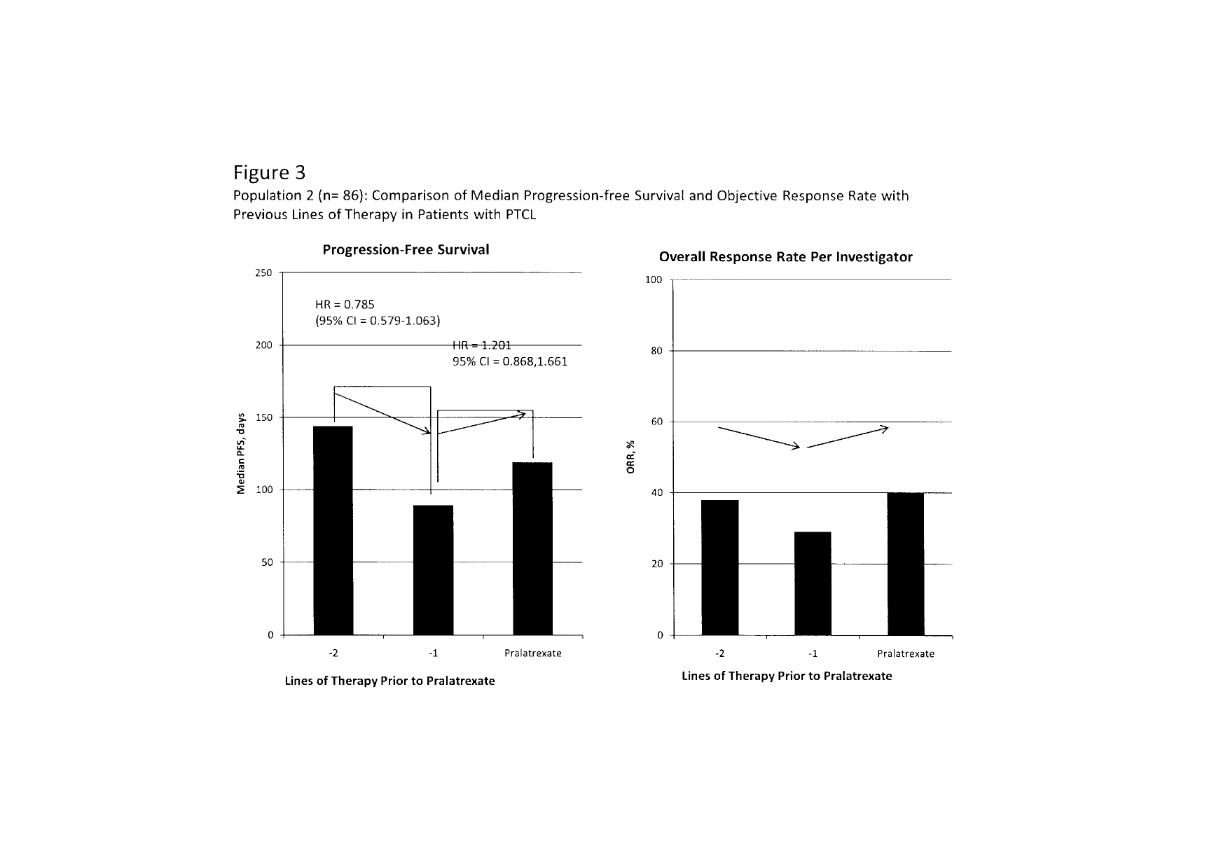

In order to examine the robustness of the pattern seen (hypothesis #1), the same analyses were performed on those patients who had undergone at least 2 previous treatments. There were 86 patients who had undergone at least 2 prior therapies before entry into PDX-008. This cohort included the 57 patients who received 3 or more previous treatments and 29 additional patients who received exactly 2 treatments prior to treatment with pralatrexate. Results are presented in Table 2. See also FIG. 3, analysis of data from the 86 patients who had undergone ≧2 previous treatments also demonstrated a trend for decreased PFS and ORR with each consecutive treatment. (FIG. 3) Once again, this trend of decreased PFS and ORR with successive lines of therapy was reversed by pralatrexate treatment, demonstrated by a higher ORR (40%) and longer PFS (median=119 days) than the previous line of therapy [HR for −1 vs pralatrexate=1.201 (0.868, 1.6661)].

For examining hypothesis #2, PFS and response rate were compared following pralatrexate treatment with the treatment immediately prior to study entry for all 109 PTCL patients evaluated for efficacy in PDX-008. This cohort included the 86 patients who received 2 or more previous treatments and 23 patients who received only 1 treatment prior to pralatrexate. Results are presented in Table 3.

Overview of Calculations: Time to progression of disease (PD) was calculated from the start date of the prior therapy administered to the date of progression+1 day.

There were prior systemic treatments that had an incomplete PD date or some PD dates were not provided by the investigators, since patients had not progressed on that treatment. There were also some incomplete dates on the start of the prior therapy treatment, but no date was completely missing. In order to calculate the time to PD for each of the prior therapies, the following assumptions were used:

Progression: If any patient's date of PD to systemic therapy was incomplete, the date was imputed to the middle of whatever the most complete data received from the case report form (CRF) (ie, if day was missing but month and year were present, the day became “15”. If both day and month were missing and year was present, their PD date was 1 July of the year reported). For patients with a completely missing PD date (day, month, and year), the PD date was set to the PD date of any subsequent therapy if it exists, otherwise it was set to date of pralatrexate initiation. If during the imputation, the assigned PD date was prior to the stop date of that treatment and the original PD date was either completely missing or missing both day and month of PD, then the PD date was imputed to the next PD date, if it exists, or to the start of pralatrexate. This approach is extremely conservative, and if anything, should lengthen the PFS for those patients in whom the relevant data points are missing.

Prior Treatment Start Date: If any patient's start date of prior systemic therapy was incomplete, the date was imputed to the middle of whatever the most complete data received from the CRF (i.e., if day was missing but month and year were present, the day became “15”. If both day and month were missing and year was present, their start date was 1 July of the year reported). No patients had a completely missing prior therapy start date. If the imputed start date of treatment is after the stop date and the progression date equaled the stop date, then the imputed start date was set to the treatment stop date.

These rules are commonly used for partial or completely missing dates of data such as adverse events, concomitant medication and medical history (Adams, 2010).

PFS on Prior Treatments:

PFS time was calculated as the number of days from start of prior treatment to the date of PD (date of PD−start date of prior treatment+1).

PFS on Pralatrexate:

PFS time was calculated as the number of days from start of treatment to the date of PD or death, regardless of cause (date of PD or death−start date of pralatrexate treatment+1).

The Kaplan-Meier estimates of the median time to PFS are presented in the tables below. In addition, the PFS hazard ratio (HR), along with 95% confidence intervals (CIs), were estimated via the Cox regression model.

Results. Analysis of data from the 57 patients who had undergone at least 3 previous treatments demonstrates a trend for PFS and response rate to decrease with each consecutive treatment. The largest decrease in PFS and response rate was observed between the third and second previous treatments. A factor in this observation may be the effect of 23 treatment-naive patients present in the third previous treatment group (−3) and none in the second or subsequent previous treatment groups, coupled with a greater response to first treatment and subsequent development of progressive resistance. This trend of reduced PFS and response rate with successive lines of therapy is reversed with pralatrexate treatment (Table 1).

TABLE 1
Comparison of Median Progression-free Survival with Previous
treatments of PTCL: Patients with ≧3 Previous Treatments

| Prior | | | Progression-free Survival | | Re- |
|---|---|---|---|---|---|
| Systemic | | | Median (Days) | Hazard Ratio | sponse |
| Treatment | Comparison | N | 95% CI | 95% CI | Rate |
| Pralatrexate | | 57 | 134.00 | | 40 |
| | | | (51.00, 162.00) | | |
| −1 | −1 vs | 57 | 95.00 | 1.120 | 30 |
| | pralatrexate | | (72.00, 136.00) | (0.752, | |
| | | | | 1.666) | |
| −2 | −2 vs −1 | 57 | 140.00 | 0.823 | 33 |
| | | | (75.00, 208.00) | (0.566, | |
| | | | | 1.195) | |
| −3 | −3 vs −2 | 57 | 213.5 | 0.660 | 56 |
| | | | (122, 280) | (0.450, | |
| | | | | 0.967) | |

A decrease in efficacy in both PFS and response rate between the second and first (immediately prior) previous treatments was also observed in the 86 patients who had undergone at least 2 previous treatments (Table 2). The trend for PFS and response rate to decrease with each subsequent treatment again is reversed with pralatrexate, despite 29 treatment-naive patients in the −2 treatment group and none in the −1 or pralatrexate treatment groups.

TABLE 2
Comparison of Median Progression-free Survival with Previous
treatments of PTCL: Patients with ≧2 Previous Treatments

| | | | Progression-free Survival | | |
|---|---|---|---|---|---|
| Prior | | | | Hazard | |
| Systemic | | | Median (Days) | Ratio | Response |
| Treatment | Comparison | N | 95% CI | 95% CI | Rate |
| Pralatrexate | | 86 | 119.00 | | 40 |
| | | | (77.00, 146.00) | | |
| −1 | −1 vs | 86 | 89.50 | 1.201 | 29 |
| | pralatrexate | | (72.00, 123.00) | (0.868, | |
| | | | | 1.661) | |
| −2 | −2 vs −1 | 86 | 144.00 | 0.785 | 38 |
| | | | (91.00, 188.00) | (0.579, | |
| | | | | 1.063) | |

When these endpoints were analyzed for all 109 PTCL patients in PDX-008 who received pralatrexate with their immediate prior treatment, a slight improvement is observed in PFS (Table 3). Based on the observations presented above (Table. 1 and Table 2) of a continual decrease in response rate, it would be expected that the response rate would also decline in this analyses and yet it remains unchanged.

TABLE 3
Comparison of Median Progression-free Survival and Response
Rate between Pralatrexate and the Immediate Prior Treatment

| | | | Progression-free | | |
|---|---|---|---|---|---|
| | | | Survival | | |
| Prior | | | | Hazard | |
| Systemic | | | Median (Days) | Ratio | Response |
| Treatment | Comparison | N | 95% CI | 95% CI | Rate |
| Pralatrexate | | 109 | 121.00 | | 39 |
| | | | (77.00, 148.00) | | |
| −1 | −1 vs | 109 | 114.00 | 1.051 | 38 |
| | pralatrexate | | (89.00, 151.00) | (0.785, | |
| | | | | 1.407) | |

TABLE 4
Populations for Progressive Resistance Analysis and Patients' Most
Recent Prior Systemic Therapy

| | Patients | Patients | Patients |
|---|---|---|---|
| | with <3 | with ≧2 | with ≧1 |
| | Prior | Prior | Prior |
| | Systemic | Systemic | Systemic |
| | Therapies | Therapies | Therapies |
| | n = 57 | n = 86 | n = 109 |
| Type of Systemic Therapy | n (%) | n (%) | n (%) |
| CHOP | 5 (9) | 13 (15) | 28 (26) |
| Platinum-containing multi- | 10 (18) | 20 (23) | 22 (20) |
| agent chemotherapy | | | |
| Non-platinum-containing | 13 (23) | 16 (19) | 19 (17) |
| multi-agent chemotherapy | | | |
| Hyper CVAD | 1 (2) | 1 (1) | 2 (2) |
| Single agent chemotherapy | 16 (28) | 19 (22) | 21 (19) |
| Stem cell transplant | 8 (14) | 9 (10) | 9 (8) |
| Bexarotene | 5 (9) | 7 (8) | 8 (7) |
| Denileukin diftitox | 2 (4) | 2 (2) | 2 (2) |
| Other | 1 (2) | 2 (2) | 2 (2) |
| Systemic investigational | 1 (2) | 2 (2) | 2 (2) |
| agents | | | |
| Steroids alone | 0 (0) | 1 (1) | 1 (<1) |

To assess the objective response rate (ORR) and PFS with each subsequent line of therapy and the effect of pralatrexate on ORR and PFS: In the first population group (n=57), i.e., group including patients with ≧3 prior systemic therapies, PFS and ORR of the third therapy prior to pralatrexate (−3) were compared with those of the second prior therapy (−2) to pralatrexate; PFS and ORR of the second prior therapy (2) were compared with those of the most recent line of therapy (−1) prior to pralatrexate; and PFS and ORR of the most recent line of therapy (−1) were compared with pralatrexate therapy for these patients.

In the second population group (n=86), i.e., group including patients with ≧2 prior systemic therapies, PFS and ORR of the second prior therapy (2) were compared with those of the most recent line of therapy (−1) prior to pralatrexate; and PFS and ORR of the most recent therapy (−1) were compared with pralatrexate therapy for these patients.

In the third population group (n=109), i.e., group including patients with ≧1 prior systemic therapy, PFS and ORR of the most recent therapy (−1) were compared with pralatrexate therapy for these patients.

Overview of Calculations: ORR: Defined as the sum of the total number of complete responses (CR), complete responses unconfirmed (CRu) and partial responses (PR) divided by the total number of patients. PFS on prior treatments: The number of days from start of prior treatment to the date of progressive disease (PD) (date of PD−start date of prior treatment+1). PFS on pralatrexate: The number of days from start of pralatrexate treatment to the date of PD or death (date of PD or death−start date of pralatrexate treatment+1). If a patient's start date of prior systemic therapy was incomplete, the date was imputed to the middle of the most complete data that was available (i.e., if day was missing but the month and year were present, the day became 15th day of that month. If both day and month were missing, but the year was present, the start date was July 1 of the year reported. No patients had a completely missing prior therapy start date. If using the method resulted in an imputed start date of treatment that was after the reported stop date and the progression date equaled the stop date, then the imputed start date was set at the treatment stop date.

Conclusion. A progressive resistance to consecutive treatments, as described in the literature for other malignancies, is shown in patients with relapsed or refractory PTCL when consecutive treatments prior to entry into PDX-008 are analyzed. Pralatrexate may stabilize or reverse this trend. Patients had a median of 3 prior systemic therapies (range 1-12). Overall in PROPEL, pralatrexate demonstrated a 39% ORR by investigator assessment and 29% by central review. The median duration of response was 8.1 months by investigator assessment and 10.1 by central review. The median duration of PFS was 4.0 months by investigator assessment and 3.5 months by central review. 57 patients had undergone at least three prior systemic therapies before entry into PROPEL. Of these 57 patients, 34 had ≧3 previous treatments and 23 had exactly 3 previous treatments. As presented in the Tables, a trend of reduced PFS and ORR with successive lines of therapy was observed. The hazard ratio (HR) for outcomes worsens with successive lines of therapy [−3 v −2: HR 0.660 (0.450, 0.967); −2 V −1:HR 0.823 (0.566, 1.195)]. Thus, patients with ≧3 prior lines of therapy (−3) had greater response rates and PFS vs. the RR and PFS in the same patients with earlier lines of therapy (−2 or −1). The trend was reversed with pralatrexate treatment demonstrated by a higher response rate (40%) and longer PFS (median=134 days) than the previous line of therapy. The only hazard ratio >1 in this analysis, indicating a longer PFS for a more recent line of therapy vs. the most recent prior line of therapy, is for pralatrexate vs.−1. The same analyses were performed on the 86 patients who had undergone at least 2 previous treatments. The trend for PFS and response rate to decrease with each subsequent treatment was again demonstrated and was again reversed with pralatrexate (HR for PFS=1.201 [−1 vs. 1] vs 0.785 [−2 vs −1]). This is the first analysis to demonstrate that patients with PTCL exhibit the same pattern of progressive resistance as seen in most other tumor types. It is also the first to demonstrate that a drug, pralatrexate, can reverse the pattern of progressive resistance in patients with drug resistant PTCL. Pralatrexate demonstrated higher responses and longer PFS than would be expected in a later of line of therapy setting, thus reversing the trend of progressive resistance.

Furthermore, the efficacy of pralatrexate is confirmed in PDX-008 when compared with the efficacy demonstrated in response to the treatment immediately prior to entry into the study. This is despite the fact that there were no treatment-naive patients in the pralatrexate treatment group but the prior treatment group included 23 treatment naive patients and 59% of patients in this group received multi-agent chemotherapy, with all the consequent implications to toxicity and quality of life. The patients in this prior treatment group are the same patients as studied in PDX-008 and represent a heterogeneous control group reflecting clinical practice. Pralatrexate demonstrated higher responses and longer PFS than would be expected in a later line of therapy, thus reversing the trend of progressive resistance. Patients enrolled in PROPEL were previously heavily pretreated with multi-agent chemotherapy (CHOP, platinum-based and non-platinum-based), single agent chemotherapy (bexarotene, denileukin diftitox, other). 9 patients had received autologous stem cell transplants immediately prior to treatment with pralatrexate; 63% of the enrolled patients had no evidence of response to their most recent prior therapy; and 24% had no evidence of response to any prior therapy.

Despite being heavily pretreated with either multi-agent or single-agent therapies, or having received a stem-cell transplant, patients in this population showed improvements in ORR and PFS after treatment with pralatrexate.

This indicated that single-agent pralatrexate had the potential to provide improved efficacy in patients with relapsed or refractory PTCL despite failure of prior therapies.

### Example 3

### Pralatrexate is Effective in Patients with Relapsed/Refractory Peripheral T-cell Lymphoma (PTCL) Following Treatment with Ifosfamide, Carboplatin, and Etoposide (ICE)-BASED REGIMENS

Background:

The prognosis of aggressive PTCL is dismal. Investigators have developed salvage regimens attempting to improve outcomes for patients with relapsed or refractory disease. One such regimen frequently used is ifosfamide, carboplatin, and etoposide (ICE) or ICE-based regimens (eg, and rituximab-ICE [RICE] and dexamethasome-ICE [DICE]). These regimens can have response rates approaching 70% and patients may proceed to a stem cell transplant, yet most patients tend to relapse quickly (Horwitz et al Blood 2005; 106:a2679). Thus, optimal approaches for patients with relapsed or refractory disease are needed, and agents based on a unique mechanism of action warrant study. This analysis was conducted to determine if pralatrexate offered a beneficial treatment option for patients after treatment with an ICE/RICE/DICE regimen. Methods: Pralatrexate was administered via intravenous push over 3 to 5 minutes at 30 mg/m²/week for six weeks in 7-week cycles with concurrent 1 mg vitamin B₁₂ intramuscular every 8 to 10 weeks and folic acid 1.0 to 1.25 mg by mouth once daily. 109 patients were treated with pralatrexate in the PROPEL study. All patients in the PROPEL study had received at least 1 prior therapy for PTCL. A subset of 20 patients had received ICE, RICE or DICE at some point prior to treatment with pralatrexate. The efficacy in these patients was compared to the remainder of the patient population who had not received ICE/RICE/DICE as a therapy for PTCL prior to pralatrexate (n=89). The patient population that previously received an ICE-based regimen was heavily pretreated with am edian of 3 prior systemic regimens (range, 2-11). The median age was 45 years, a younger patient population compared with the overall PROPEL population (59 years).

TABLE 5
Patient baseline demographics (N = 20)

| | Patients |
|---|---|
| Parameter | n (%) |
| Gender | |
| Male | 13 (66) |
| Female | 7 (35) |
| Age | |
| <65 | 14 (70) |
| ≧65 | 6 (30) |
| Median age, years (range) | 45 (21-76) |
| Race | |
| White | 13 (66) |
| Black | 3 (15) |
| Asian | 2 (10) |
| Hispanic | 1 (5) |
| Unknown | 1 (5) |
| Histopathology | |
| PTCL unspecified | 11 (55) |
| Anaplastic large cell lymphoma | 6 (30) |
| Angioimmunoblastic T-cell lymphoma | 2 (10) |
| Blastic NK lymphoma (with skin, lymph node, | 1 (5) |
| or visceral involvement) | |
| ECOG PS | |
| 0 | 7 (35) |
| 1 | 8 (40) |
| 2 | 5 (25) |
| Number of prior systemic regimens, median (range) | 3 (2-11) |
| 2 | 5 (25) |
| 3 | 7 (35) |
| 4 | 2 (10) |
| ≧5 | 6 (30) |

The median time from diagnosis was 13.9 months (range, 4.2 to 118.8 months). Most common histopathologies in patients who were previously treated with an ICE-based regimen were PTCL unspecified, anaplastic large cell lymphoma, and angioimmunoblastic T-cell lymphoma. Five patients (25%) had a response to an ICE-based regimen prior to pralatrexate therapy. Complete response (CR)=3 (15%), partial response (PR)=2(10%), stable disease (SD)=15%, progressive disease (PD)=7 (35%), not assessable (NA)=5 (25%).

ORR, duration of response, and progression-free survival (PFS) by independent central review and investigator assessment, and OS, are presented in Table 6. Details of the patients who responded to pralatrexate according to assessment by independent central review are provided in Table 7.

TABLE 6
Summary of Efficacy Results (N = 20)

| | | Independent | Investigator |
|---|---|---|---|
| | | Central Review | Assessment |
| Efficacy Assessments | | n (%) | n (%) |
| Tumor response | ORR (CR + PR) | 8 (40) | 8 (40) |
| | CR | 3 (15) | 5 (25) |
| | PR | 5 (25) | 3 (15) |
| | SD | 2 (10) | 5 (25) |
| | PD | 4 (20) | 6 (30) |
| | NA | 6 (30) [symbol] | 1 (5) |

| | | |
|---|---|---|
| Duration of response | | |
| Median,^{b} months (95% CI) | 13.1 (3.4, NA) | 16.2 (3.4, NA) |
| Range, months [symbol] | 1.3-22.1 | 1 day-22.2 |
| PFS | | |
| Median,^{b} months (95% CI) | 14.4 (1.5, NA) | 4.8 (2.5, 14.4) |
| Range, months [symbol] | 1 day-23.6 | 1 day-23.8 |

| | |
|---|---|
| OS | |
| Median,^{b} months (95% CI) | 12.0 (5.2, 23.4) |
| Range, months | 1.1-24.0^{d} |

CI = confidence interval.
^{a}Two patients were not evaluable and 4 were missing as they discontinued treatment in cycle 1 without a response assessment.
^{b}Median per a Kaplan-Meier estimate.
^{c}Patients without PD were censored for duration of response at the date of last response assessment before the end of treatment or at day 1 (for a duration of 1 day) if there were no follow up response assessments, and for PFS at least response assessment or first dose if there were no response assessments (for PFS of 1 day).
^{d}Survival follow up ended at two years. [symbol]
indicates data missing or illegible when filed

TABLE 7
Details of Patients previously treated with ICE who
responded to pralatrexate (N = 8).

| Response to | | Duration | | Response |
|---|---|---|---|---|
| Pralatrexate | | of | | to |
| per Independent | | Response | PFS | ICE-based |
| Central Review | Histology | (months) | (months) | Regimen |
| PR | PTCL | 22.1 [symbol] | 23.6 [symbol] | NA |
| | unspecified | | | |
| CR | PTCL | 17.1^{b} | 18.6 [symbol] | PD |
| | unspecified | | | |
| PR | PTCL | 13.1 | 14.4 | SD |
| | unspecified | | | |
| PR | Anaplastic large | 10.1 | 17.8 | CR |
| | cell lymphoma | | | |
| CR | Anaplastic large | 4.9 [symbol] | 8.3 [symbol] | NA |
| | cell lymphoma | | | |
| PR | Anaplastic large | 3.4 | 4.8 | PR |
| | cell lymphoma | | | |
| PR | PTCL | 1.6 | 3.0 | PD |
| | unspecified | | | |
| CR | Anaplastic large | 1.3 [symbol] | 5.9 [symbol] | SD |
| | cell lymphoma | | | |

NA = not assessable.
Patients without PD were censored for duration of response at the date of last response assessment before the end of treatment or at day 1 (for a duration of 1 day) if there were no follow-up response assessments, and for PFS at last response assessment or first dose if there were no response assessments (for PFS of 1 day).
Calculations censored due to: ^{a}patient still in response at time of data cut-off, ^{b}response assessments no longer required or ^{c}stem cell transplant. [symbol]
indicates data missing or illegible when filed

Results:

The demographics and disease characteristics in the 20 patients with prior ICE/RICE/DICE were reflective of the overall patient population. The majority of patients had PTCL-unspecified. The overall response rate (ORR) to pralatrexate in the 20 patients according to independent central review using International Workshop Criteria was 40% (15% complete responses [CR]) compared with 27% (10% CR/complete response unconfirmed [CRu]) for the other 89 patients. The ORR based on investigators assessment was 40% (25% CR/CRu) for the 20 patients compared to 39% (17% CR/CRu) for the other patients. The median duration of response to pralatrexate in the 20 patients was 13.1 months for response assessed by central review (versus 9.4 months for the other 89 patients), and was even longer based on response per investigator assessment 16.2 months (versus 6.8 months for the other 89 patients). Median progression-free survival (PFS) in the 20 patients was over 3 times that of the other patients: PFS according to central review=14.4 months versus 3.3 months. Of the 15 patients who did not have a response to a prior ICE-based regimen, six (40%) responded to pralatrexate. Nine of 20 patients received ICE-regimens as their most recent therapy prior to pralatrexate. Only one of these 9 patients responded to ICE. Of the 8 patients who did not respond to this classic salvage combination chemotherapy, 3 did respond to pralatrexate (2 CR and 1 pr). The 20 patients who were previously treated with ICE/RICE/DICE stayed on treatment with pralatrexate longer than the rest of the patient population. These 20 patients received a median of 11 doses of pralatrexate and were on treatment for a median of 139 days compared to the remaining 89 patients who received a median of 7 doses and were treated for a median of 64 days. Two of the 20 patients previously treated with ICE/RICE/DICE prior to treatment with pralatrexate proceeded to a stem cell transplant upon attaining a response to pralatrexate. The duration of response to pralatrexate was censored for these 2 patients (at 1.3 and 4.9 months). However, additional follow-up has demonstrated that the current disease-free period (duration of response: pralatrexate+transplant) for these 2 patients, who both still have a CR, is 10.9 and 30.8 months. Conclusions: Patients treated with ICE/RICE/DICE have very limited effective treatment options if their disease is refractory or they have relapsed. Patients treated with prior ICE/RICE/DICE were able to maintain extended treatment with pralatrexate, and had a high level of response and extensive PFS.

### Example 4

### Pralatrexate is Effective as Second-Line Treatment Following CHOP in Patients with Relapsed/Refractory Peripheral T-cell Lymphoma (PTCL)

Background:

The most common therapy for first-line treatment of PTCL is the multi-agent combination cyclophosphamide/doxorubicin/vincristine prednisone (CHOP). However, most patients progress within 6-12 months, and there is no standard of care for second-line treatment of these patients. Thus optimal second-line approaches are needed and agents based on a unique mechanism of action warrant study. This analysis was conducted to determine if pralatrexate offered a beneficial second-line treatment option. Methods: 109 patients were treated with pralatrexate in the PROPEL study (O'Connor 0, Pro B, Pinter-Brown L, et al. Pralatrexate in patients with relapsed or refractory peripheral T-Cell lymphoma (PTCL): results from the pivotal PROPEL study. J Clin Oncol 2011 29(9):1182-1189). All patients in the PROPEL study had received at least 1 prior therapy for PTCL. A subset of 15 patients had received CHOP as their only therapy prior to treatment with pralatrexate. The efficacy in these patients was compared with the remainder of the patient population who had not received CHOP as their only therapy for PTCL prior to pralatrexate (n=94).

Data:

Baseline disease characteristics and demographics for the 15 patients who received CHOP as first-line therapy were similar to the overall PROPEL patient population (Table 8)

TABLE 8
Patient Characteristics in this Subgroup

| Parameter, n (%) | Patients (N = 15) |
|---|---|
| Histopathology (per central review) | |
| PTCL-u | 8 (53) |
| Anaplastic large cell lymphoma | 3 (20) |
| T/NK nasal type | 2 (13) |
| Angioimmunoblastic | 1 (7) |
| Transformed mycosis fungoides | 1 (7) |
| Gender | |
| Male | 9 (60) |
| Female | 6 (40) |
| Age, years | |
| <65 | 9 (60) |
| ≧65 | 6 (40) |
| Median age, years (range) | 60 (30-71) |
| ECOG PS | |
| 0 | 8 (53) |
| 1 | 7 (47) |
| Bone marrow involvement | |
| Positive | 4 (27) |
| Negative | 10 (67) |
| Indeterminate | 1 (8) |
| Skin involvement | |
| Yes | 6 (40) |
| No | 9 (60) |
| Extranodal growth | |
| Yes | 11 (73) |
| No | 4 (27) |
| Lactose dehydrogenase | |
| Normal | 7 (47) |
| High | 8 (53) |

ECOG PS, Eastern Cooperative Oncology Group Performance Status.

Eleven patients (73%) had a response to CHOP as first-line therapy prior to pralatrexate treatment (6 with complete response [CR] and 5 with partial response [PR]; Table 9.

TABLE 9
Best Response to First-Line CHOP

| Best Response, n (%) | Patients (N = 15) |
|---|---|
| CR | 6 (40) |
| PR | 5 (33) |
| SD | 1 (7) |
| PD | 3 (20) |

PD, progressive disease;
SD, stable disease.

The median time from initial diagnosis to entry in the PROPEL study for this subgroup was 16 months (range, 3.1-82.3). The median time since progression from first-line CHOP treatment prior to entry in the PROPEL study was 1.1 months (range, 0.3-57.1

Efficacy Results With Second-Line Pralatrexate for Patients Who Received First-Line CHOP: Overall response rate (ORR), duration of response, progression-free survival (PFS), by independent central review and by investigator assessment, and overall survival (OS) are presented in Table 10.

TABLE 10
Summary of Efficacy Results for This Subset After
Second-Line Pralatrexate

| | | Central Review | Investigator |
|---|---|---|---|
| Efficacy Assessments, n (%) | | (N = 15) | (N = 15) |
| Tumor Response | ORR | 7 (47) | 6 (40) |
| | (CR + CRu + PR) | | |
| | CR | 3 (20) | 4 (27) |
| | CRu | 0 (0) | 1 (7) |
| | PR | 4 (27) | 1 (7) |
| | SD | 4 (27) | 4 (27) |
| | PD | 4 (27) | 4 (27) |
| | Not evaluable | 0 (0) | 1 (7)^{a} |

| | | |
|---|---|---|
| Duration of Response | | |
| Median, months (95% CI) | NE^{b} (6.5, NE) | 12.5 (10.8, NE) |
| Range, months | 1 day^{c}-10.1^{c} | 2.1^{c}-16.1^{c} |
| PFS | | |
| Median, months (95% CI) | 8.1 (4.6, NE) | 7.4 (2.3, 15.0) |
| Range, months | 0.7-17.8^{c} | 1 day^{c}-17.8^{c} |

| | |
|---|---|
| OS | |
| Median, months (95% CI) | NE^{b} (9.0, NE) |
| Range, months | 1.5-24.0^{d} |

Note:
Medians per Kaplan-Meier estimate.
^{a}Not evaluable as patient discontinued treatment in cycle 1 without a response assessment;
^{b}NE = not able to estimate as there were insufficient PD events at the time of last follow-up;
^{c}Patients without PD were censored for duration of response at the date of last response assessment before the end of treatment or at day 1 (for a duration of 1 day) if there were no follow-up response assessments, and for PFS at last response assessment or first dose if there were no response assessments (for PFS of 1 day);
^{d}Survival follow-up ended at 2 years.
CI, confidence interval;
CRu, CR unconfirmed;
PD, progressive disease;
SD, stable disease.

A median OS time was not able to be estimated as 11 (73%) of the 15 patients were still alive at the time of last contact; the 12-month OS estimate was 73%.

TABLE 11
Details of Patients Who Responded to Pralatrexate as Second-Line
Therapy After First-Line CHOP

| | | Results With Pralatrexate | | |
|---|---|---|---|---|
| Response | | (Per Independent Central Review) | | |
| to | | | Duration of | |
| First-Line | | | Response | PFS |
| CHOP | Histology | Response | (months) | (months) |
| SD | T/NK-cell | CR | 10.1^{a} | 14.8^{a} |
| | lymphoma-nasal | | | |
| CR | Angioimmunoblastic | CR | 9.7^{b} | 17.8^{b} |
| | T-cell lymphoma | | | |
| PR | T/NK-cell | PR | 8.3^{b} | 15.5^{b} |
| | lymphoma-nasal | | | |
| CR | PTCL-u | PR | 6.5^{a} | 8.1 |
| CR | PTCL-u | CR | 3.3^{c} | 4.6^{c} |
| PD | PTCL-u | PR | 2.3^{c} | 3.4^{c} |
| PR | PTCL-u | PR | 1 day^{a} | 4.6^{a} |

Patients without progressive disease (PD) were censored for duration of response at the date of last response assessment before the end of treatment or at day 1 (for a duration of 1 day) if there were no follow-up response assessments, and for PFS at last response assessment or first dose if there were no response assessments (for PFS of 1 day). Calculations censored due to ^{a}initiation of other subsequent therapy, ^{b}in response and continuing pralatrexate therapy, or ^{c}stem cell transplant. SD, stable disease. Pralatrexate demonstrated a high level of response in this subgroup as assessed by independent central review (47%) and the study investigators (40%)

Responses were durable, with a median duration of response of 12.5 months per investigator assessment, and not reached per central review. Two of the 15 patients remained on treatment (time on treatment=12.9 and 18.5 months) and in response as of the data cut-off, and therefore their duration of response data were censored. One of these patients was characterized as having T/NK-cell lymphoma-nasal subtype and the other had angioimmunoblastic T-cell lymphoma. Two patients proceeded to stem cell transplant after response to pralatrexate and thus were censored for duration of response (at 2.3 and 3.3 months). These 2 patients remained in CR at the time of last contact, and their current disease-free period (duration of response to pralatrexate+time since stem cell transplant) is 20.9 and 27.2 months. Both of these patients were characterized as having PTCLu and had received CHOP×6 as their first-line therapy prior to pralatrexate; details of these patients are as shown below:

| | Patient 1 | Patient 2 |
|---|---|---|
| Response to first-line CHOP | PD | CR |
| Response to second-line | PR | CR |
| pralatrexate | | |
| Stem cell transplant | Allogeneic | Autologous |

Overall Results:

The demographics and disease characteristics in the 15 patients with prior CHOP were reflective of the overall patient population. The majority of patients had PTCL-unspecified. The overall response rate (ORR) to pralatrexate in the 15 patients according to independent central review using International Workshop Criteria was 47% (20% complete responses [CR]) compared with 27% (10% CR/complete response unconfirmed [CRu]) for the other patients in the study. The ORR based on investigators assessment was 40% (33% CR/CRu) for the 15 patients compared to 39% (16% CR/CRu) for the other patients. The median duration of response to pralatrexate in the 15 patients is not yet estimable for response assessed by central review, but was longer than that of the other patients in the study based on response per investigator assessment: 12.5 months versus 6.7 months. Median progression-free survival (PFS) in the 15 patients was over twice that of the other patients: PFS according to central review=8.1 months versus 3.1 months, PFS based on response per investigator assessment=7.4 months versus 3.5 months. The 15 patients who were previously treated only with CHOP stayed on treatment with pralatrexate longer than the rest of the patient population. The 15 patients received a median of 16 doses of pralatrexate and were on treatment for a median of 134 days compared to the other 94 patients who received a median of 7 doses and were treated for a median of 67 days. Two of the 4 patients who remained on treatment as of the data cut-off were from the group of 15 patients treated with first-line CHOP. Two of the 15 patients treated only with CHOP prior to treatment with pralatrexate proceeded to a stem cell transplant upon attaining a response to pralatrexate. The duration of response to pralatrexate was censored for these 2 patients (at 2.3 and 3.3 months). However, additional follow-up has demonstrated that the current disease-free period (duration of response: pralatrexate+transplant) for these 2 patients, who both still have a CR, is 20.1 and 21.7 months. Conclusions: Patients who were previously treated only with CHOP were able to maintain extended second-line treatment with pralatrexate, and had a high level of response and a greatly increased PFS. These results may reflect a mechanistic rationale for the CHOP-pralatrexate sequence (ie, alkylator-antifolate). Second line pralatrexate may lead to increased opportunities for transplantation approaches with curative intent for some patients. Pralatrexate is highly effective as a single agent and should be considered as a second-line therapeutic option for relapsed or refractory PTCL.

### Example 5

Characteristics and Outcomes for Patients with Relapsed/Refractory Peripheral T-cell Lymphoma (PTCL) Treated with <1 and ≧1 or ≧2 Cycles of Pralatrexate

Background:

The pivotal PROPEL study of pralatrexate (FOLOTYN®) in patients with relapsed or refractory PTCL reported a rapid response to treatment in a heavily pretreated patient population. The majority of patients (63% of responders) responded by their first assessment at 7 weeks (the end of cycle 1). The present analysis assessed if there were any unique characteristics of patients who were unable to complete the first cycle of therapy and to assess the outcomes of patients who received ≧1 or ≧2 cycles of pralatrexate. Pralatrexate, a folate analog targeting dihydrofolate reductase, was approved in the United States for treatment of relapsed or refractory PTCL based on the PROPEL study. Methods: In PROPEL, patients received 30 mg/m² pralatrexate once weekly for 6 weeks in successive 7-week cycles. This analysis evaluates the efficacy and safety of pralatrexate in patients who did not complete 1 cycle (n=45) compared to patients who completed either ≧1 (n=64) or ≧2 (n=43) cycles of treatment. Cycle completion was determined receiving ≧1 dose in the following cycle. Thus, the subgroup of patients who completed ≧1 (n=64) includes those patients who only completed 1 cycle+the 43 who completed ≧2 cycles of treatment. Results: The demographics of patients who did not complete cycle 1 were similar to the overall population in PROPEL. However, the patients not completing cycle lwere more likely to have the angioimmunoblastic subtype (20% vs 6%). Patients in each subgroup received a median of 3 prior treatment regimens. However, patients who did not complete cycle 1 had a shorter interval between disease diagnosis and study entry suggesting that these patients progress more rapidly, with a median time since initial diagnosis of 12.2 months as compared to either 19.6 months or 21.8 months for the ≧1 and ≧2 subgroups, respectively. Objective response rate (ORR) by independent central review was greater for patients who completed either ≧1 (45%) or ≧2 (54%) cycles compared to patients who did not complete cycle 1 (7%). A summary of the efficacy analyses are presented in the table (Table 12) below.

TABLE 12

| | Treatment | Treatment | Treatment |
|---|---|---|---|
| | Duration | Duration | Duration |
| | <1 cycle | ≧1 cycle | ≧2 cycles |
| ORR by Central Review | 7% | 45% | 54% |
| (CR + CRu + PR) | | | |
| ORR by Investigator Assessment | 7% | 63% | 70% |
| (CR + CRu + PR) | | | |
| Median Duration of Response by | Not | 10.1 | 13.1 |
| Central Review (months)* | estimable | | |
| Median PFS by Central Review | 1.3 | 4.9 | 8.1 |
| (months)** | | | |
| Median OS (months) | 8.2 | 20.5 | 22.9 |

*insufficient events occurred to estimate the median based on response by central review
**per a Kaplan-Meier estimate
CR = complete response
CRu = CR unconfirmed
PR = partial response
PFS = progression-free survival

Of the 45 patients who did not complete cycle 1, 62% discontinued due to progressive disease (PD), 29% due to adverse events (AEs), 2% due to stem cell transplant after achieving a response to pralatrexate and 7% for other reasons. The most frequent reasons for treatment discontinuation were similar for patients with <1 cycle, ≧1 cycle or ≧2 cycles, although the incidence of discontinuation was lower for patients with ≧1 or 2 cycles. The overall incidence and severity of AEs was generally similar across subgroups, though patients who did not complete cycle 1 reported a slightly higher incidence of Grade 3-4 thrombocytopenia (<1: 40%; ≧1: 28%; ≧2: 21%) and anemia (<1: 27%; ≧1: 13%; ≧2: 9%). The incidence of Grade 3-4 mucosal inflammation was higher in patients who did not complete cycle 1 (<1: 24%, ≧1: 19%, ≧2: 12%). However, the percentage of patients in whom mucosal inflammation led to treatment discontinuation was similar (<1: 4%; ≧1: 6%; ≧2: 2%). Eight patients (13%) had dose reductions due to mucosal inflammation in cycle 1, were able to continue treatment further, and 4 of the 8 had a response to pralatrexate. An additional 4 patients (9%) had dose reductions in cycle 2 due to mucosal inflammation, were able to continue treatment, and 3 of the 4 had a response to pralatrexate. Conclusions: As would be expected, patients who received ≧1 or ≧2 cycles of pralatrexate had better outcomes than those patients who did not complete cycle 1. Patients who were unable to complete cycle 1 were more likely to have quickly progressed through multiple lines of therapy and were more likely to have the angioimmunoblastic subtype, which a subtype is thought to have a different biology than other PTCL subtypes and lower chemosensitivity. Further analysis of this subtype is warranted.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method to reverse the trend towards progressive resistance to consecutive treatments for T-cell lymphoma in a patient, comprising: selecting a patient having T-cell lymphoma, wherein the patient has had at least one prior treatment for T-cell lymphoma, wherein the at least one prior treatment comprises non-10-propargyl-10-deazaminopterin treatment; and administering to the patient a composition comprising a therapeutically effective amount of 10-propargyl-10-deazaminopterin, whereby the trend towards progressive resistance is reversed.

2. The method of claim 1, wherein the trend towards reversal of progressive resistance to consecutive treatments for T-cell lymphoma in a patient is measured by extension of the number of days of the patient's progression-free survival upon treatment with a composition comprising 10-propargyl-10-deazaminopterin, relative to the patient's number of days of progression-free survival for the immediately-prior non-10-propargyl-10-deazaminopterin treatment.

3. The method of claim 1, wherein the 10-propargyl-10-deazaminopterin is substantially free of 10-deazaminopterin.

4. The method of claim 1, wherein the T-cell lymphoma is peripheral T-cell lymphoma.

5. The method of claim 1, wherein the T-cell lymphoma is cutaneous T-cell lymphoma.

6. The method of claim 1, wherein the T-cell lymphoma is a subcutaneous panniculitic T-cell lymphoma.

7. The method of claim 1, wherein the T-cell lymphoma is a human T-lymphotropic virus (HTLV)-associated T-cell lymphoma/leukemia.

8. The method of claim 1, wherein the T-cell lymphoma is selected from: (a) lymphoblastic lymphomas in which the malignancy occurs in primitive lymphoid progenitors from the thymus; (b) mature or peripheral T cell neoplasms, including T cell prolymphocytic leukemia, T-cell granular lymphocytic leukemia, aggressive NK-cell leukemia, cutaneous T cell lymphoma (Mycosis fungoides/Sezary syndrome), anaplastic large cell lymphoma, T cell type, enteropathy-type T cell lymphoma, Adult T-cell leukemia/lymphoma including those associated with HTLV-1, and angioimmunoblastic T cell lymphoma, and subcutaneous panniculitic T cell lymphoma; and (c) peripheral T cell lymphomas that initially involve a lymph node paracortex and never grow into a true follicular pattern.

9. The method of claim 1, wherein the 10-propargyl-10-deazaminopterin is in a salt form.

10. The method of claim 1, wherein the composition comprising 10-propargyl-10-deazaminopterin is administered in an amount of from 30 to 275 mg/m² per dose.

11. The method of claim 10, wherein the composition comprising 10-propargyl-10-deazaminopterin is administered weekly.

12. The method of claim 10, wherein the composition comprising 10-propargyl-10-deazaminopterin is administered in an amount of 30 mg/m² per dose.

13. The method of claim 10, wherein the composition comprising 10-propargyl-10-deazaminopterin is administered biweekly.

14. The method of claim 10, wherein the composition comprising 10-propargyl-10-deazaminopterin is administered in one or more cycles, each cycle comprising administration once weekly for six weeks in an amount of from 30 to 150 mg/m² per dose followed by a one week rest.

15. The method of claim 1, wherein the composition is administered as an oral liquid or as an injectable solution.

15. The method of claim 1, further comprising supplementation with folic acid and vitamin B₁₂.

16. The method of claim 1, wherein the patient has had one prior non-10-propargyl-10-deazaminopterin treatment.

17. The method of claim 1, wherein the patient has had two prior non-10-propargyl-10-deazaminopterin treatments.

18. The method of claim 1, wherein the patient has had three prior non-10-propargyl-10-deazaminopterin treatments.

19. The method of claim 1, wherein the patient has had greater than three prior non-10-propargyl-10-deazaminopterin treatments.

20. The method of claim 1, wherein the at least one prior non-10-propargyl-10-deazaminopterin treatment comprises CHOP.

21. The method of claim 1, wherein the at least one prior non-10-propargyl-10-deazaminopterin treatment comprises ICE.

22. The method of claim 1, wherein the T-cell lymphoma is relapsed/refractory T-cell lymphoma.
